(19) United States
(12) Patent Application Publication
(10) Pub. No.: US 2007/0249862 A1
(43) Pub. Date: Oct. 25, 2007

(54) Process for the Production of Polyisocyanate/Polysilicic Acid Based Resins with Widely Variable Processability and Setting Periods

(76) Inventors: Gabor Nagy, Budapest (HU); Ferenc Balazs, Bugyi (HU); Gyorgy Banhegy, Budapest (HU); Miklos Barothy, Budapest (HU)

Correspondence Address: NATH & ASSOCIATES, 112 South West Street, Alexandria, VA, 22314, US

(21) Appl. No.: 11/660,279

(22) PCT Filed: Aug. 17, 2005
(86) PCT No.: PCT/HU05/00089

§ 371 Date: May 29, 2007

(30) Foreign Application Priority Data
Aug. 19, 2004 (HU) P 0401708

(51) Int. Cl. C07C 55/24

(52) U.S. Cl. 562/590

(57) ABSTRACT

The invention relates to a method for the production of polyisocyanate/polysilicic acid based resins with widely variable processability period and setting period by reacting one or more polyisocyanates with water glass optionally in the presence of one or more additives and/or auxiliary agents conventionally used in the production of polyisocyanate/polysilicic acid based resins. According to the invention the reaction is performed in the presence of one or more cocatalysts comprising mobile hydrogen of acidic character, wherein the cocatalyst is a compound comprising a structural unit of formula (I) a metal chelate thereof or a derivative thereof in which one of the mobile hydrogens of acidic character is replaced by a substituent X, wherein X represents halo, hydrocarbyl, hydrocarbyl-oxy, hydrocarbyl-carbonyl, hydrocarbyl-oxy-carbonyl or hydrocarbyl-carbonyl-amido group or a combined group formed from two or more of these groups, and the cocatalyst is used in an amount of at least 0.01% by weight calculated for the combined weight of polyisocyate(s) and water glass.

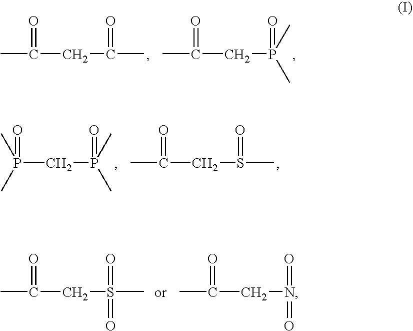

The invention relates to a process for the production of polyisocyanate/polysilicic acid based resins the processability period (in other words: gelling period or pot life) and setting period (in other words: hardening or curing period) of which can be varied within wide limits.

Combined polyisocyanate/polysilicic acid resin systems, prepared by reacting polyisocyanates (also including diisocyanates) with water glass, have been elaborated initially to replace Freon®-comprising polyurethane foams. They have become well-known in the late seventies mainly from the works of Dietrich, and numerous variants of them have been elaborated since then [see e.g. Polyurethane, Chapter 2.4.9 in G. W. Becker and D. Braun: Kunststoff Handbuch 7 (K. Hansen Verlag, München, 1983), furthermore HU 168 856, HU 169 478, HU 176 469, HU 207 746 and HU 208 330]. In these resins most of the relatively sensitive urethane bonds, characteristic of polyurethanes, are replaced by much more stable isocyanurate rings formed in a trimerisation reaction of three terminal isocyanate groups, and the thus-formed polyisocyanurate matrix surrounds the polysilicic acid gel particles. Owing to the reaction of isocyanate with water the matrix comprises a substantial amount of polyurea derivatives, too, which are also more stable than the urethane bonds. The main catalyst of these reactions is the alkali present in water glass. However, in order to obtain products with good mechanical characteristics, cocatalysts—e.g. trimerisating catalysts well known in polyurethane chemistry, particularly tertiary amines [see e.g. Behrend, G., Dedlet, J.: Plaste und Kautschuk 3, 177-180 (1976); Kreste, J. E., Hsieh, K. H.: Makromol. Chem. 178, 2779-2782 (1978); U.S. Pat. No. 4,540,781]—and/or various active diluents (HU 207 746 and HU208 330) are required which reduce the processability period of the resin-forming composition to some minutes. This short processability period is disadvantageous in several fields of use, because it renders difficult or even sometimes impossible to incorporate various filling agents into the resin matrix, and leaves a very short time for shaping and fitting the resin composition and for correcting optional failures.

The method disclosed in HU 212 033 and in the respective US and German patents (U.S. Pat. No. 5,622,999; DE 4 121 153) represented a breakthrough in this field. According to this method various phosphorous acid esters have been used as cocatalysts or reactive diluents, whereupon the processability period of the resin-forming composition has become variable within wide limits but the mechanical strength charasteristics of the end-product have remained acceptable. In this method phosphorous acid esters have been frequently used together with various amines in order to attain a more fine adjustment of processability period.

However, from the aspects of environment protection it is objectionable that most of the phosphorous acid esters used as cocatalysts and of the optionally added amines can be partially leached out with water from the crosslinked matrix; thus products made of such materials which contact with great amounts of water (e.g. pipelines, container linings etc.) may represent a potential risk to natural environment and to the living world of waters. It is also disadvantageous that certain phosphorous acid esters, particularly derivatives with higher molecular weights, also act as plasticizers, impairing thereby the mechanical strength of the end-product when used in amounts required to adjust the processability period to the desired value. In certain fields of use (e.g. for preparing pipe linings) users require resin-forming compositions with relatively long processability periods which, when once shaped and fitted, harden within a very short time. This demand can only rarely be satisfied with compositions comprising phosphorous acid ester cocatalysts.

Now it has been found that when compounds comprising mobile hydrogen of acidic character, more particularly, compounds comprising 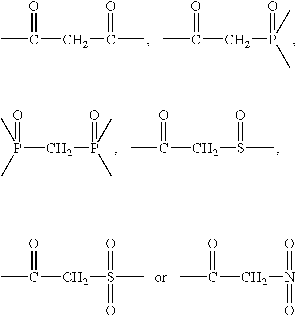
structural units (these are termed in the following as “AMH compounds”), metal chelates thereof or derivatives thereof in which one of the mobile hydrogens is replaced by an X substituent (these are termed in the following as “substituted AMH compounds”) are used as cocatalysts in the production of polyisocyanate/polysilicic acid based resins, the processability period of the polyisocyanate/polysilicic acid based resins can be varied and controlled within very wide limits, the setting time of the resin-forming composition can be reduced considerably sometimes even when their processability period is relatively long, and resins can be obtained which are better in final mechanical properties than those obtained with phosphorous acid ester type cocatalysts. The cocatalysts to be used according to the invention are extensively harmless to environment and health, and do not require the use of amine compounds for the fine adjustment of processability period. Thus, using such cocatalysts, amine compounds which represent a potential damage to the environment can be fully excluded. It is not required to use phosphorous acid ester cocatalysts together with the cocatalysts to be used according to the invention. If, however, a phosphorous acid ester cocatalyst disclosed in the patents cited above is also added to the resin-forming mixture for adjusting the processability period more accurately, its required amount can be lowered substantially in comparison to that used in the known solution. Thus the potential environmental damages arising from the use of phosphorous acid esters can also be fully excluded or at least considerably reduced.

Based on the above, the invention relates to a method for the production of polyisocyanate/polysilicic acid based resins with widely variable processability period and setting period by reacting one or more polyisocyanates with water glass optionally in the presence of one or more additives and/or auxiliary agents conventionally used in the production of polyisocyanate/polysilicic acid based resins. According to the invention the reaction is performed in the presence of one or more cocatalysts comprising mobile hydrogen of acidic character, wherein the cocatalyst is a compound comprising a structural unit of 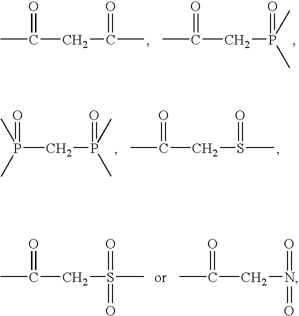
a metal chelate thereof or a derivative thereof in which one of the mobile hydrogens of acidic character is replaced by a substituent X, wherein X represents halo, hydrocarbyl, hydrocarbyl-oxy, hydrocarbyl-carbonyl, hydrocarbyl-oxy-carbonyl or hydrocarbyl-carbonyl-amido group or a combined group formed from two or more of these groups, and the cocatalyst is used in an amount of at least 0.01% by weight calculated for the combined weight of polyisocyate(s) and water glass.

The term “hydrocarbyl” as used in the definition of group X covers alkyl, alkenyl, alkynyl, cycloalkyl, cycloalkenyl and homocyclic aryl groups and combined groups formed from two or more of these groups (examples of such combined groups are arylalkyl and cycloalkyl-alkyl groups. These hydrocarbyl groups may bear optionally one or more non isocyanate-reactive substituents. The term “hydrocarbyl-carbonyl-amido group” covers formamido group, too. Of the combined groups which may stand for X hydrocarbyl groups bearing a halo, hydrocarbyl-oxy, hydrocarbyl-carbonyl or hydrocarbyl-carbonyl-oxy substituent are mentioned as examples.

Compounds comprising a 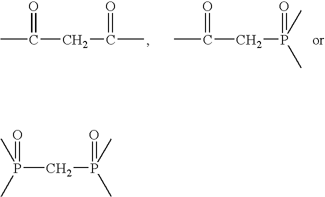
structural unit, metal chelates thereof and derivatives thereof wherein one of the acidic mobile hydrogen is replaced by substituent X form a preferred group of cocatalysts usable in the process according to the invention.

Of the cocatalysts the following compound types proved to be particularly preferred: α,β-diketones, cyclic α,β-diketones, α,β,γ-triketones, esters of α,β-keto-carboxylic acids, amides of α,β-ketocarboxylic acids, esters of cyclic α,β-ketocarboxylic acids, esters of α,β,β-diketo-monocarboxylic acids, mixed esters of α,β-ketocarboxylic acids and vinylcarboxylic acids formed with glycols, esters of α,β-ketocarboxylic acids formed with polyols, diesters of α,β-dicarboxylic acids, diamides of α,β-dicarboxylic acids, cyclic esters of α,β-dicarboxylic acids, oligo- or polyesters formed from α,β-dicarboxylic acids and polyols, oligo- or polyesters with terminal ester or ether groups also comprising α,β-dicarboxylic acid units, diesters of 3-oxo-dicarboxylic acids, diesters of α,γ-acetylated dicarboxylic acids, α,β-ketophosphonates, α,β-ketoacid ester phosphonates, α,β-diphosphonates, metal chelates of the compounds listed above, derivatives of the compounds listed above wherein one of the hydrogens of the —CH₂— group is replaced by substituent X, furthermore malonic acid, acetoacetic acid, acetylacetone and derivatives thereof.

Particularly preferred representatives of the cocatalysts are the following compounds:

compounds of formula (I)
R—CO—CHY—CO—R¹ (I)
wherein

Y is hydrogen, halo, phenyl, C1-C6 alkyl, C2-C6 alkenyl, C1-C6 alkoxy, phenyl-(C1-C6 alkyl), (C1-C6 alkoxy)-(C1-C6 alkyl), (C1-C6 alkoxy)-carbonyl, form-amido, (C1-C6 alkyl)-carbonyl-amido or (C1-C6 alkyl)-carbonyl, and

R and R¹ each stand for

(i) C1-C10 alkyl bearing optionally one or more halo, (C1-C6 alkyl)-carbonyl or (C1-C6 alkoxy)-carbonyl substituent(s),

(ii) phenyl group or a monocyclic heteroaryl group comprising a single hetero atom, each bearing optionally one or more C1-C6 alkyl or C1-C6 alkoxy substituent(s),

(iii) a group of formula —OR², wherein R² stands for hydrogen, a metal atom, phenyl-(C1-C6 alkyl), C2-C10 alkenyl or a C1-C15 alkyl bearing optionally one or more hydroxy, C1-C6 alkoxy, (C1-C6 alkyl)-CO-(C1-C6 alkyl)-CO—O— or (C2-C6 alkenyl)-carbonyl-oxy substituent(s),

(iv) a group of formula —NR³R⁴ wherein R³ and R⁴ each stand for hydrogen, C1-C6 alkyl or hydroxy-(C1-C6 alkyl) or one of them may also represent phenyl wherein the phenyl group may optionally bear an alkyl-carbonyl-alkyl-carbonyl-amido substituent;

(v) C2-C10 alkenyl which may optionally bear a phenyl substituent and the phenyl group may optionally bear a hydroxy and/or C1-C6 alkoxy substituent; or

one of R and R¹ may also represent halo, (C1-C6 alkoxy)-carbonyl, a residue of a polyol wherein the hydroxy groups are esterified and/or etherified, or a group of the formulae —O-(Alk-O)ₘ-CO-Alk¹-CO-Alk², —O-(Alk-O—CO-Alk¹-CO)ₙ-O-Alk², —O-(Alk-O)ₙ-CO-Alk² or —O-(Alk-O)ₙ-(Alk¹-O)ₙ-Alk², and in these latter formulae m is 1-2500, n is 1-60, Alk² is C1-C6 alkyl or C2-C6 alkenyl and Alk and Alk¹ stand for C1-C6 alkylene which may be the same or different; or

R and R¹ may form together a —NH—CO—NH— group, a C2-C4 alkylene chain or a methylenedioxy group, all of which may optionally bear one or more C1-C6 alkyl substituent(s), or

R and Y may form together a C2-C4 alkylene chain or a C2-C4 alkylene-oxy chain, all of which may optionally bear one or more C1-C6 alkyl substituents;

metal chelates of compounds of formula (I) wherein Y is hydrogen and R and R¹ are as defined in points (i) and (ii) above; furthermore

compounds of formulae (II) and (III), 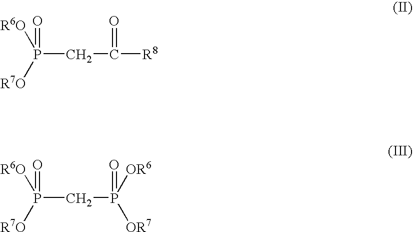
wherein R⁶ and R⁷ each stand for C1-C6 alkyl which may be the same or different and R⁸ represents C1-C6 alkyl, C1-C6 alkoxy or phenyl.

Of the cocatalysts listed above acetoacetic esters and acetylacetone derivatives with flash points above 60° C., preferably above 100° C., form an other particularly preferred group with the further advantage that they improve the adhesion between metal, ceramic and enamel surfaces and resin coatings contacting therewith.

Characteristic representatives of cocatalysts to be used according to the invention are the compounds listed in Tables 1 and 2. The cocatalysts listed in Table 1 are compatible with polyisocyanates, thus they can also be introduced as master batches formed with polyisocyanates. The cocatalysts listed in Table 2 are either compatible with water glass, thus they can also be introduced as master batches formed with water glass, or they are incompatible with both water glass and polyisocyanates, thus they should be introduced either separately or as master batches formed with additives and/or auxiliary agents which are compatible with the particular compound. It should be mentioned here that the relatively inexpensive representatives of cocatalysts produced on large scale are sometimes less pure than the highest analytical purity grade. These more or less contaminated cocatalysts of industrial quality are also suitable for the purpose of the invention. However, for the preparation of master batches in such instances it should be taken into account that the impurities may occasionally decrease the storability of master batches formed with water glass or polyisocyanates. If a water glass or polyisocyanate master batch comprising a cocatalyst of industrial quality is not storable without quality change for at least 6 months, it is preferred to add the cocatalyst of industrial quality to the reaction mixture either as a separate component or as a master batch formed with compatible additives and/or auxiliary agents (e.g. plasticizers, diluents etc.) which is storable without quality change for at least 6 months.

The cocatalysts listed above are used in an amount of at least 0.01% by weight, preferably at least 0.05% by weight, related to the combined weight of water glass and polyisocyanate(s). The upper limit of the amount of cocatalysts is of lower importance and depends essentially on the chemical structure of the particular cocatalyst (or cocatalyst mixture) and on the processability period to be attained. As an information, when the less active substituted AMH compounds are used as cocatalysts (optionally combined with AMH compounds or metal chelates thereof), the upper limit of the amount of cocatalysts may be 30% by weight, related to the combined weight of water glass and polyisocyanate(s), or even higher; namely the substituted AMH compounds also act as plasticizers and thus they may replace a part or the whole amount of plasticizers to be used. In such instances, however, it is usually not required to use the cocatalyst in an amount exceeding 40%. If only AMH compounds or metal chelates thereof are used as cocatalysts, much lower amounts than those given above are also sufficient; the upper limit of the amount of such cocatalysts may be usually 0.5-5% by weight related to the combined weight of water glass and polyisocyanate(s).

To prepare the polyisocyanate/polysilicic acid based resins according to the invention any of the water glass and polyisocyanate types (this latter term comprises diisocyanates, too) used in the preparation of known polyisocyanate/polysilicic acid systems can be used.

Preferred representatives of water glass are solutions of various Li, Na and K water glasses with a SiO₂/M₂O modulus (M=Li, Na or K) of 1.8 to 2.8 and with a viscosity at room temperature (η_{22° C.}) of 50 to 2000, preferably of 200 to 1000. Mixtures of different water glass types can also be used in the method of the invention.

Preferred representatives of polyisocyanates (also comprising diisocyanates) are the compounds listed below and any mixtures thereof:

The MDI (methylene-diphenyl-diisocyanate) compound family, the most important member of polyurethane chemistry, should be mentioned at the first place, because this is produced worldwide in the highest amounts. Commercially available members of this family are the so-called monomeric MDI's, which comprise two aromatic rings, such as pure 4,4′-MDI, various mixtures of 4,4′-MDI and 2,4′-MDI, furthermore the more complex isomeric and oligomeric mixtures, such as the crude MDI (CR-MDI) series. The viscosity of the members of this latter series varies within about from 150 mPa.s to about 3000 mPa.s. In the variants of higher viscosities the ratio of oligomeric MDI with three or more rings gradually increases, thus they are also termed in the literature as polymeric MDI's (P-MDI). P-MDI types can be used particularly preferably in the process of the invention.

Starting from monomeric MDI's numerous prepolymers are produced by the industry, which are also applicable in the process of the invention. These are diisocyanates prepared from polyether and/or polyester polyols wherein one MDI molecule, each, has been reacted with the two terminal —OH groups. The properties of the resin can be controlled within wide limits by the appropriate selection of the molecular weight (chain length) and/or chemical structure of the starting polyol. Longer polyol chains (Mₙ≧1500) are particularly suitable to increase flexibility and ductility of the product. It should be noted here that polyether prepolymers formed from polyols, when used in higher amounts, also exert cocatalytic effects; despite of this fact these compounds are classified here not into the group of optionally used other cocatalysts but into the group of polyisocyanates.

Several so-called modified MDI types are available where 5-25% of the monomeric or oligomeric MDI molecules have been reacted or converted; these are marketed as solutions formed with the excess of the starting oligomeric MDI. MDI types comprising carbodiimide bonds (CD-MDI) and MDI's coupled to polyisocyanate rings by trimerisation (PIR-MDI) are of great importance. The last mentioned ones are suitable for the production of products with increased resistance to heat and chemicals.

Over the various MDI types tolylene-diisocyanate (TDI) and prepolymers formed therefrom analogously to the MDI derivatives can also be used in the process of the invention.

The weight ratio of polyisocyanates to water glass may vary within the limits well known from the literature. The polyisocyanate:water glass weight ratio may be generally 1:(0.1-1.5), preferably 1:(0.2-1), particularly 1:(0.3-0.8).

As it has already been mentioned before, the reaction of polyisocyanates and water glass can be performed optionally in the presence of one or more additives and/or auxiliary agents conventionally used in the preparation of polyisocyanate/polysilicic acid resins. These additives and/or auxiliary agents may be those listed in the above references, of which the following ones are mentioned as examples: borax, mono- and polyols, plasticizers, diluents, fire retardants, antifoaming agents, adhesion-increasing agents, tixotropic agents, thickeners, pigments, colourants, mono-, di- or polyester-type compounds which are partially or fully built into the resin matrix, tenzides, etc. Their amounts may vary within the ranges known from the literature. Furthermore, other cocatalysts known from the literature (e.g. from the references cited above) can optionally also be used to obtain a more fine adjustment of the processability period and/or setting period; their amounts are, however, usually much less than the usual ones.

In all instances where any of the cocatalysts, additives and auxiliary agents comprises one or more unsaturated bonds capable of radical polymerization, it is preferred to use an inorganic and/or organic free radical initiator (characteristic representatives of which are the peroxy compounds) as additive, too, the amount of which is generally up to 3% by weight calculated for the total weight of the polyisocyanates plus water glass. The free radical initiator can be introduced either as a separate component or as a master batch formed with additional components which are compatible with the initiator. When a free radical initiator is used, the strength of the cross-linked product can be increased, and simultaneously the amount of organic components which can be extracted or leached out from the matrix can be reduced considerably.

According to our experiences AMH compounds and metal chelates thereof ensure a relatively quick gelling, whereas substituted AMH compounds ensure a relatively slow gelling. By the appropriate selection of such cocatalysts or by the appropriate combination of the two types (AMH compound or metal chelate thereof on one hand and substituted AMH compound on the other hand) the processability period of the resin-forming components can be controlled within very wide limits. When using an appropriate combination of AMH compounds or metal chelates thereof and substituted AMH compounds sometimes no plasticizer is required. When the cocatalyst is an AMH compound or a metal chelate thereof and a relatively long processability period is to be attained, preferably a plasticizer and/or an other cocatalyst ensuring a more prolonged processability period should also be added to the mixture. Preferred representatives of said other cocatalysts are phosphorous acid esters of higher molecular weights disclosed in HU 212 033, of which amounts much lower than that required in the absence of an AMH compound are sufficient. When the cocatalyst is a substituted AMH compound and the processability period attainable using this compound should be shortened, it is preferred to add to the mixture an other known cocatalyst which ensures short processability period. As it has already been mentioned before, it is not advisable to use amines for this purpose; or when still an amine is used for any reason, its amount must be kept within the ranges allowed by the provisions of environment protection.

According to a preferred method two or more master batches, storable for at least 6 months without a change in quality, are prepared from the reactants to be used in the process of the invention, and the resin is formed directly at the place of utilization by admixing the master batches in appropriate ratios. Of them master batch “A” comprises water glass in admixture with other water glass-compatible additives; master batch “B” comprises the polyisocyanates in admixture with other polyisocyanate-compatible additives; and when the cocatalyst to be used in the process of the invention cannot be placed in any of master batches “A” and “B”, a separate master batch (master batch “C”) is also formed which comprises the cocatalyst in admixture with a part of additives and/or auxiliary agents compatible with it. At the place of use master batches “A”, “B” (and optionally “C”) are admixed with one another in prescribed volume ratios. Such master batches have been used in the examples which illustrate further details of the process of the invention. In the examples the compositions of the individual master batches are given as weight percentages, and the mixing ratios of the master batches are given as a volume ratios. The densities required to recalculate these figures are given in the examples.

To prepare the test pieces appropriate amounts of master batches “A”, “B” (and optionally “C”) are poured first into a laboratory beaker and stirred intensely for 1 minute with a laboratory spatula made of stainless metal. The mixture is then set aside and the processability period (also termed as pot life which is the shortest period elapsed until pourability ceases) is determined. These values are listed in Table 4.

In the knowledge of processability periods further mixtures are prepared which are poured some minutes before termination of processability period into metal moulds lubricated with a mould release agent. Five test pieces, 20×20×120 mm in dimensions, are prepared in each of the moulds.

Next day the moulds are dismounted and one day and one week after pouring the test pieces are subjected to a known three-point bending/tensile test using a support length of 100 mm and a speed of 100 mm/min, which latter much exceeds the usual one. The compressive forces measured on one day and one week old test pieces are listed in Tables 4 and 5.

Percentage compositions of the master batches used in the individual examples and mixing ratios of the master batches are summarized in Table 3.

### EXAMPLES 1 to 13

Master batches of the following compositions were used as starting materials:

Master batch “A” (weight: 155 g; volume: 100 ml)
100% by weight of Betol 3P type sodium water glass produced by Woeliner Silikat (Ludwigshafen, GFR). The characteristics of the liquid water glass are as follows: modulus: 2.0, viscosity at 20° C.: 600 mPa.s, density: 1.55 g/cm³ Master batch “B” (weight: 300 g; volume: 250 ml)
Reactive components (termed in TAble 3 as “reactives”):
70% by weight of Ongronat CR 30-40 type polymeric MDI produced by Borsodchem Rt (Kazincbarcika, Hungary) with an isocyanate content of 31% by weight, viscosity at 20° C.: 400 mPa.s, density: 1.23 g/cm³
5% by weight of TA-52 type MDI-based polyether prepolymer produced by Polinvent Kft (Budapest, Hungary) with an isocyanate content of 8% by weight, viscosity at 40° C.: 3000 mPa.s, density: 1.15 g/cm³
   Additives:
15% by weight of Disflammol type diphenyl-cresyl phosphate produced by Bayer (a known phosphoric acid ester type plasticizer and flame retardant; in this instance cocatalyst, too), density: 1.20 g/cm³
10% by weight of 1-acetyl-naphthalene produced by Merck (diluent); density:

1.12 g/cm³

When preparing the reference test piece master batch “A” was admixed with master batch “B” (this is reference example No. 01 in Tables 3 and 4).

The cocatalysts used according to the invention in the individual examples are listed in Table 4 under the heading “compounds comprising acidic mobile H”. These were used in two different amounts in all of the examples. In variants (a) the amount of cocatalyst in the whole reaction mass was 6.0 g (2.0% by weight calculated for the total weight of master batch “B”; about 1.6% by weight calculated for the combined weight of water glass and polyisocyanates), whereas in variants (b) the amount of cocatalyst in the whole reaction mass was 0.6 g (0.2% by weight calculated for the total weight of master batch “B”; about 0.16% by weight calculated for the combined weight of water glass and polyisocyanates).

In Example 1 the cocatalyst was placed into master batch “A” so that the amount of cocatalyst was completed to 155 g (=100 ml) with Betol 3P and the thus completed master batch “A” (the cocatalyst content of which is given in Table 3) was admixed with 250 ml (=300 g) of master batch “B”.

In Examples 2 to 11 the cocatalyst was placed into master batch “B” so that the amount of cocatalyst was completed to 300 g (=250 ml) with master batch “B” of the above composition, and the thus completed master batch “B” (the cocatalyst content of which is given in Table 3) was admixed with 155 g (=100 ml) of Betol 3P.

In Examples 12 and 13 the cocatalyst was not compatible with any of master batches “A” and “B”. Therefore a mixture of additives was separated from the additives of master batch “B”, the amount of cocatalyst was completed to 55 g (=50 ml) with the separated mixture of additives, and the resulting master batch “C” (the cocatalyst content of which is given in Table 3) was admixed with the liquid mixture containig the remainder of master batch “B” (245 g=200 ml) and with 155 g (=100 ml) of Betol P.

The processability periods (in Table 4: ref. pot life) of the resin-forming mixtures and the bending/tensile forces measured after 1 day and 1 week, respectively, are given in Table 4. Where Table 4 does not contain data under the headings of “ref. pot life” and “bending/tensile force” this means that no measurements were performed. From the data of Table 4 it appears that the processability period of the resin forming mixture can be varied within very wide limits (from 1 minute to 2 hours) by the appropriate selection of the cocatalyst. It also appears that the cocatalysts used according to the invention always shorten considerably the setting time, even when the processability period had been adjusted to 2 hours. This follows from the fact that although the bending/tensile forces measured on 1 week old resins are always approximately the same, the tensile/bending forces measured on 1 day old samples are always much greater than those of the reference product, consequently the resins prepared according to the invention reach their final strenght within a much shorter period.

### EXAMPLES 14 to 21

Master batches of the following compositions were used as starting materials:

Master batch “A” (weight: 155 g; volume: 100 ml)
100% by weight of Betol 3P type sodium water glass produced by Woellner Silikat (Ludwigshafen, GFR). The characteristics of the liquid water glass are as follows: modulus: 2.0, viscosity at 20° C.: 600 mPa.s, density: 1.55 g/cm³ Master batch “B” (weight: 240 g; volume: 200 ml)
Reactive components (termed in Table 3 as “reactives”):
70% by weight of Ongronat CR 30-40 type polymeric MDI produced by Borsodchem Rt (Kazincbarcika, Hungary) with an isocyanate content of 31% by weight, viscosity at 20° C.: 400 mpa.s, density: 1.23 g/cm³
15% by weight of TA-52 type MDI-based polyether prepolymer produced by Polinvent Kft (Budapest, Hungary) with an isocyanate content of 8% by weight, viscosity at 40° C.: 3000 mPa.s, density: 1.15 g/cm³; in this amount it exerts cocatalytic effect, too.
   Additives:
9% by weight of 1-acetyl-naphthalene produced by Merck (diluent); density:

1.12 g/cm³
6% by weight of diallyl phthalate produced by Merck (plasticizer and diluent);

density: 1.12 g/cm³

When preparing the reference test piece master batch “A” was admixed with master batch “B” (this is reference example No. 014 in Tables 3 and 4).

The cocatalysts used according to the invention in the individual examples are listed in Table 4 under the heading “compounds comprising acidic mobile H”. Where two compounds are given in Table 4 this means that a 1:1 w/w mixture of the two compounds was used. The cocatalysts were used in two different amounts in all of the examples. In variants (a) the amount of cocatalyst in the whole reaction mass was 5.0 g (2.08% by weight calculated for the total weight of master batch “B”; about 1.6% by weight calculated for the combined weight of water glass and polyisocyanates), whereas in variants (b) the amount of cocatalyst in the whole reaction mass was 0.5 g (0.2% by weight calculated for the total weight of master batch “B”; about 0.16% by weight calculated for the combined weight of water glass and polyisocyanates).

In Example 14 the cocatalyst was placed into master batch “A” so that the amount of cocatalyst was completed to 155 g (=100 ml) with Betol 3P and the thus completed master batch “A” (the cocatalyst content of which is given in Table 3) was admixed with 200 ml (=240 g) of master batch “B”.

In Examples 15 to 19 the cocatalyst was placed into master batch “B” so that the amount of cocatalyst was completed to 240 g (=200 ml) with master batch “B” of the above composition, and the thus completed master batch “B” (the cocatalyst content of which is given in Table 3) was admixed with 155 g (=100 ml) of Betol 3P.

In Examples 20 and 21 the cocatalyst was not compatible with any of master batches “A” and “B”. Therefore 22 g (=20 ml) of an additive mixture of the above composition were separated from master batch “B”, the amount of cocatalyst was completed to 22 g (=20 ml) with the separated additive mixture, and the resulting master batch “C” (the cocatalyst content of which is given in Table 3) was admixed with the remainder of master batch “B” (218 mg=180 ml) and with 155 g (=100 ml) of Betol P.

The processability periods (in Table 4: ref. pot life) of the resin-forming mixtures and the bending/tensile forces measured after 1 day and 1 week, respectively, are given in Table 4. Where Table 4 does not contain data under the headings of “ref. pot life” and “bending/tensile force” this means that no measurements were performed. From the data of Table 4 it appears that the processability period of the resin forming mixture can be varied within sufficiently wide limits (6-30 minutes) by the appropriate selection of the cocatalyst. As these systems gellify relatively quickly, there are no considerable differences between the strength data of the test pieces measured after 1 day. It is, however, very surprising that the cocatalysts used according to the invention, even those which ensured the longest processability period, always led to the formation of products of much higher final strength.

### EXAMPLE 22

Master batches of the following compositions were homogenized with one another:

Master batch “A”: 100 ml (=155 g) of Betol 3P (see Example 1)

Master batch “B” (weight: 460 g, volume: 400 ml):
40% by weight of Ongronat CR 30-40 (see Example 1)
40% by weight of PEG 2000 bis-acetoacetate
8% by weight of tributyl phosphate
10% by weight of Disflammol DPK (see Example 1)
2% by weight of Eusolex® 9020 (see Table 1).

PEG 2000-bis-acetoacetate was prepared under big-laboratory conditions from PEG-2000 polyol and ethyl acetoacetate by transesterification at 80° C. for about 2 hours under continuous vacuum distillation. The viscosity of the product is 1800 mpa.s at 20° C.

The resin-forming mixture does not contain sufficient sodium hydroxide to bind completely and continuously the liberated carbon dioxide, which latter foamed the emulsion of rapidly increasing temperature and viscosity shortly after homogenization. The volume of the resulting foam was about the fivefold of the volume of the starting liquid. The cell structure of the foam was slightly inhomogeneous, but its compressive strength was close to that of hard polyurethane foams of similar densities.

### EXAMPLE 23

Master batches of the following compositions were homogenized with one another:

Master batch “A”: 100 ml (=155 g) of Betol 3P (see Example 1)

Master batch “B” (weight: 330 g, volume: 300 ml):
80% by weight of TDI prepolymer obtained from LGJ Bt (Budapest, Hungary). The prepolymer was produced from TDI 80/20 isocyanate mixture and PPG 2000 type polyol, its isocyanate content was 4.0, its viscosity was 4000 mPa.s at 20° C.
20% by weight of a diethyl malonate/neopentyl glycol condensate

When preparing the diethyl malonate/neopentyl glycol condensate 2 moles of diethyl malonate were reacted with 1 mole of neopentyl glycol at 80° C. for 2 hours in the presence of some tenth percent of sodium methylate; the liberated ethanol was distilled off using water jet vacuum. The viscosity of the resulting product is 2500 mPa.s at 20° C.

Shortly after homogenizing the two master batches an elastomeric foam of good quality, about 360 g/l in density, was formed, which is particularly suitable to fill up dilatation gaps in the building industry.

### EXAMPLE 24

The process described in Examples 14 to 21 was followed with the difference that a mixture of 2.5 g of diethyl-allyl-malonate and 2.5 g of Lonzamon® AAEMA (see Table 1) was used as cocatalyst which was placed into master batch “B”, and 1% by weight of sodium persulphate (a free radical initiator) was dissolved in master batch “A”. In this instance no test pieces were prepared using one tenth amount of the cocatalyst mixture. The characteristics of the resulting test pieces are given in Table 4.

Upon the effect of the free radical initiator the colour of the crosslinked test pieces darkened somewhat and got brownish. These test pieces had outstandingly good bending/tensile strength characteristics.

### EXAMPLE 25

The process described in Example 24 was followed with the difference that Fivenox B50G (1:1 w/w mixture of dibenzoyl peroxide and dicyclohexyl phthalate sold by Finomvegyszer Kft, Budapest, Hungary) was used as free radical initiator in an amount of 1% by weight calculated for the weight of master batch “B”. Like in Examples 20 and 21, this free radical initiator was added to the reaction mixture in the form of master batch “C”. The characteristics of the resulting test pieces are given in Table 4.

In this instance the colour of the crosslinked test pieces darkened only very slightly. These test pieces, again, had outstandingly good bending/tensile strength characteristics superseding even the results of the test pieces prepared using an inorganic peroxide free radical initiator.

TABLE 1
Cocatalysts compatible with polyisocyanates

| | | | Ex- | |
|---|---|---|---|---|
| Compound | | Formula of | ample | |
| group | Cocatalyst | the cocatalyst | for use | CAS No. |
| α,β-Diketones | Acetylacetone | 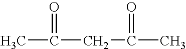 | 13. | 123-54-6 |
| | 2,2,6,6-Tetramethyl- 3,5-heptanedione | 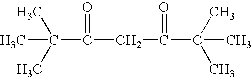 | | 1118-71-4 |
| | Benzoylacetone | 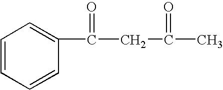 | 2. | 93-91-4 |
| | 1,3-Diphenyl-1,3- propanedione | 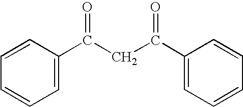 | | 120-46-7 |
| α,β-Diketones sub- stituted on the CH₂ group | 3-Chloro-acetyl- acetone | 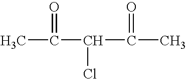 | | 1694-29-7 |
| | 3-Bromo-acetyl- acetone | 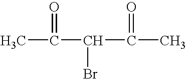 | | — |
| | 3-Methyl-acetyl- acetone | 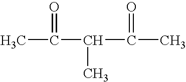 | | — |
| Other substituted α,β-diketones | 1,1,1-Trifluoro- 2,4-pentadione | 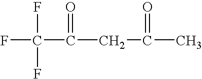 | | 367-57-7 |
| | Eusolex^{(R)} 9020 | 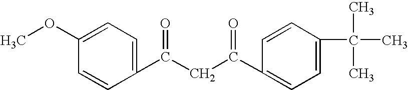 | 23. | 70356-09-1 |
| | 1-(2-Thenoyl)- 3,3,3-trifluoro- acetone | 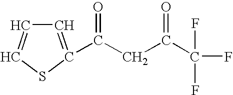 | | 326-91-0 |
| Metal chelates of α,β-diketones | Fe(III) acetyl- acetonate | 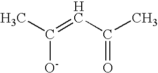 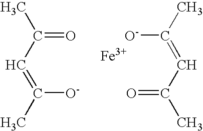 | | 14024-18-1 |
| | Cu(II) acetyl- acetonate | 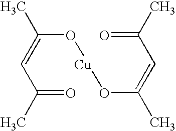 | | 13395-16-9 |
| | Mn(II) acetyl- acetonate | 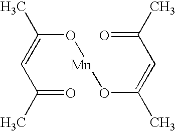 | | 14024-58-9 |
| | Zn(II) acetyl- acetonate | 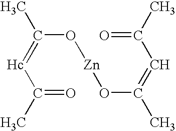 | | 14024-63-6 |
| | Zr(IV) acetyl- acetonate | 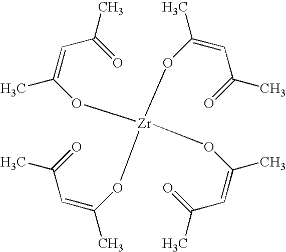 | 3. | 17501-44-9 |
| Cyclic α,β-diketones | 1,3-Cyclopen- tanedione | 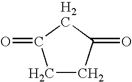 | | 3859-41-4 |
| | 1,3-Cyclo- hexanedione | 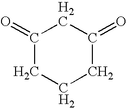 | 20. | 504-02-9 |
| | Acetyl γ-bu- tyrolactone | 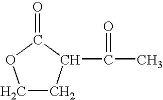 | 4. | 517-23-7 |
| | Dimedone | 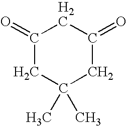 | | 126-81-8 |
| α,β,γ-Triketones | 1,3-Diacetoxy- acetone | 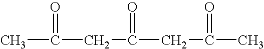 | | 6946-10-7 |
| Esters of α,β-keto acids | Methyl-aceto- acetate | 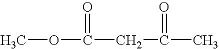 | | 105-45-3 |
| | Ethyl-aceto- acetate | 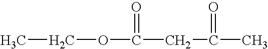 | | 141-97-9 |
| | Isopropyl acetoacetate | 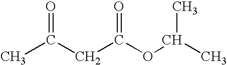 | | 542-08-5 |
| | Isobutyl acetoacetate | 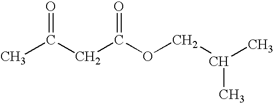 | | 7779-75-1 |
| | tert•Butyl acetoacetate | 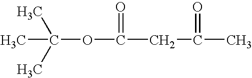 | 18. | 1694-31-1 |
| | Allyl aceto- acetate | 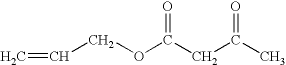 | | 1118-84-9 |
| | Benzyl aceto- acetate | 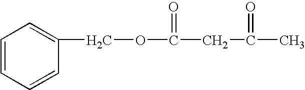 | 5 | 5396-89-4 |
| | Dodecyl aceto- acetate | 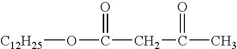 | | 52406-22-1 |
| | Acetoacetates of polyols | 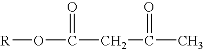 | | — |
| | Methyl oxo- pentanoate | 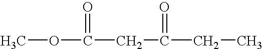 | | 30414-53-0 |
| | Ethyl 3-oxo- valerate | 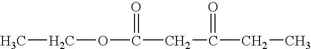 | | 4949-44-4 |
| | Ethyl butyryl- acetate | 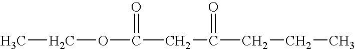 | | 3249-68-1 |
| | Methyl-isobu- tytyrlacetate | 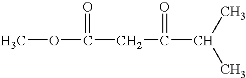 | 16. | 42558-54-3 |
| | Ethyl benzoyl- acetate | 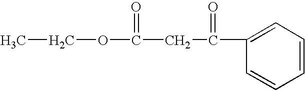 | | 94-02-0 |
| Esters of α,β-ketoacids substituted on the CH₂group | Ethyl 2-chloro- acetoacetate | 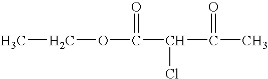 | | 609-15-4 |
| | Ethyl 2-phenyl- acetoacetate | 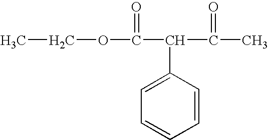 | | 5413-05-8 |
| Esters of other sub- stituted α,β-ketoacids | Ethyl 4-chloro- acetoacetate | 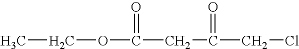 | | 638-07-3 |
| | Ethyl 4,4,4-tri- fluoroaceto- acetate | 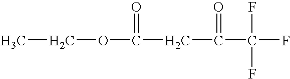 | | 383-63-1 |
| | Ethyl chloro- formylacetate | 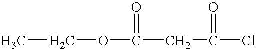 | | 36239-09-5 |
| N,N-substituted amides of α,β-ketoacids | N,N-Dimethyl acetoacetamide | 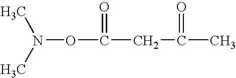 | | 2044-64-6 |
| | N,N-Diethyl acetoacetamide | 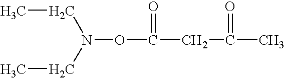 | 16. | 2235-46-3 |
| Esters of cyclic of α,β-ketoacids | Ethyl cyclopen- tanone-2-carb- oxylate | 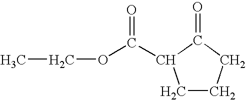 | | 611-10-9 |
| | Ethyl 2-oxo- cyclohexane- carboxylate | 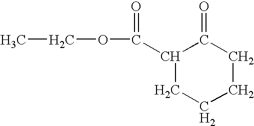 | 6. | 1655-07-8 |
| Esters of α,β,β-diketo- monocarboxylic acids | Ethyl aceto- pyruvate | 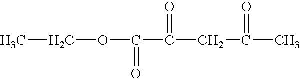 | | 615-79-2 |
| | tert•Butyl ace- topyruvate | 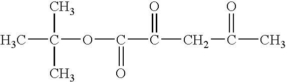 | | — |
| Mixed esters of α,β- ketoacids and vinyl- carboxylic acids formed with glycols | Lonzamon^{(R)} AAEA Acetoacetic acid ethyleneglycol acrylate | 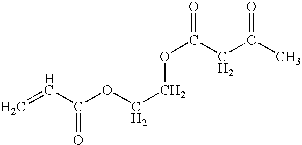 | | 21282-96-2 |
| | Lonzamon^{(R)} AAEMA Acetoacetic acid ethyleneglycol methacrylate | 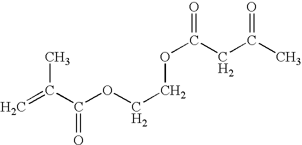 | 17. | 21282-97-3 |
| | Lonzamon^{(R)} AABUA Acetoacetic acid butyleneglycol acrylate | 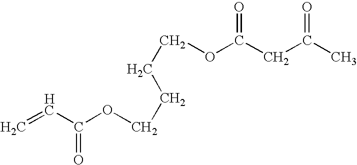 | | 13025-07-5 |
| | Lonzamon^{(R)} AAPRA 2-Acetoacetoxy- propyl acrylate and 2-Acetoacetoxy- isopropyl acrylate | 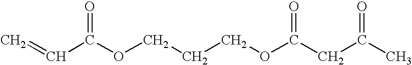 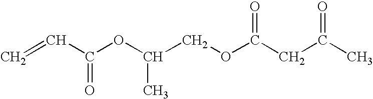 | | — |
| | Lonzamon^{(R)} AATMP Propan-1,1,1-triyl- trimethyl-tris-aceto- acetate | 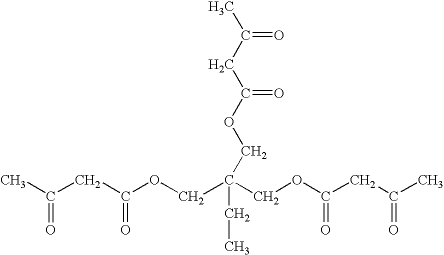 | | 22208-25-9 |
| α-β Ketosavak többérték [symbol] alkoholokkal képzett észterei | Ethyleneglycol bis(acetoacetate) | 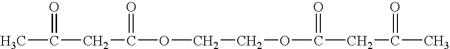 | | — |
| | PEG-2000-bis (acetoacetate) | 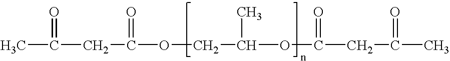 | 23. | — |
| Diesters of α,β-di- carboxylic acids | Dimethyl malonate | 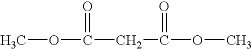 | 7. | 108-59-8 |
| | Diethyl malonate | 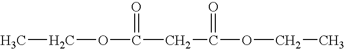 | | 105-53-3 |
| | Diisopropyl malonate | 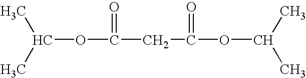 | | 13195-64-7 |
| | tert•Butyl-methyl malonate | 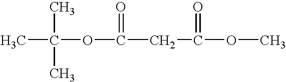 | | 42726-73-8 |
| | tert•Butyl-ethyl malonate | 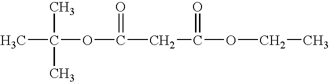 | | 32864-38-3 |
| | Dibenzyl malonate | 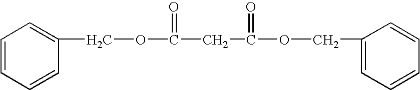 | | 15014-25-2 |
| Diesters of α,β-di- carboxylic acids substituted on the CH₂ group | Dimethyl meth- ylmalonate | 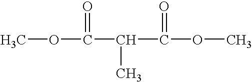 | | — |
| | Dimethyl meth- oxymalonate | 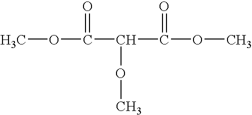 | 8. | 5018-30-4 |
| | Dimethyl allylmalonate | 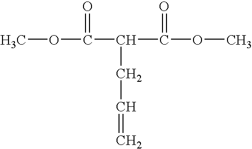 | | 40637-56-7 |
| | Dimethyl chloromalonate | 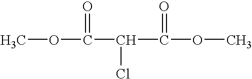 | | 28868-76-0 |
| | Diethyl methyl- malonate | 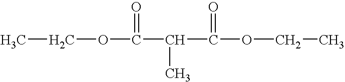 | | 609-08-5 |
| | Diethyl ethyl- malonate | 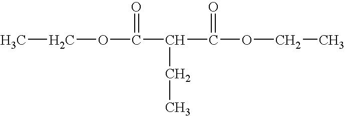 | | 133-13-1 |
| | Diethyl n-prop- ylmalonate | 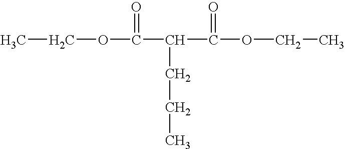 | | 2163-48-6 |
| | Diethyl iso- propylmalonate | 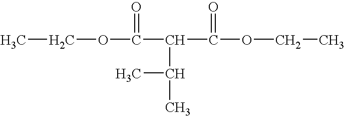 | | 759-36-4 |
| | Diethyl n-butyl- malonate | 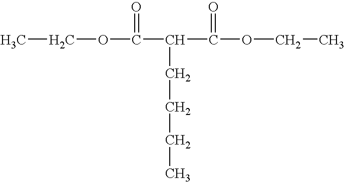 | | 133-08-4 |
| | Diethyl allyl- malonate | 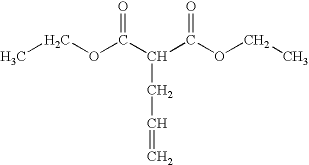 | | 2049-80-1 |
| | Diethyl ethoxy- methylmalonate | 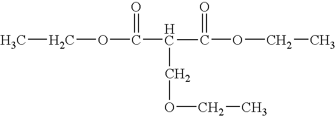 | | 87-13-8 |
| | Diethyl chloro- malonate | 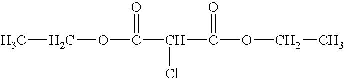 | | 14064-10-9 |
| | Diethyl phenyl- malonate | 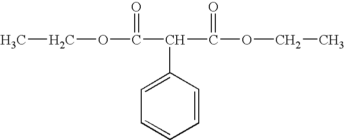 | 9. | 83-13-6 |
| | Diethyl benzyl- malonate | 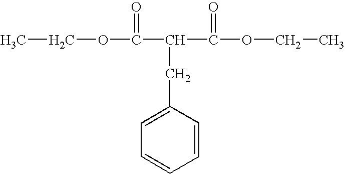 | | 607-81-8 |
| | Triethyl methane- tricarboxylate | 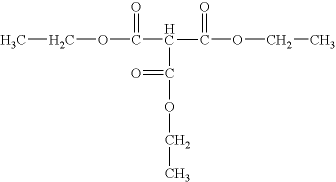 | | 6279-86-3 |
| Derivatives of α,β- dicarboxylic acids (e.g. N,N-substituted amides | Tetramethyl malonamide | 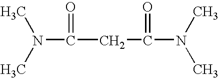 | | — |
| Cyclic esters of α,β- dicarboxylic acids | Meldrum's acid | 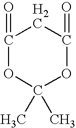 | | 2033-24-1 |
| Oligo- or polyesters prepared from α,β-di- carboxylic acid esters and polyols | Diethylmalonate ethyleneglycol condensate Mₙ =2000 | 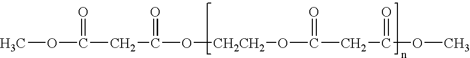 | | — |
| | Diethylmalonate neopentylglycol condensate Mₙ = 800 | 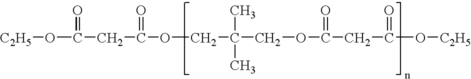 | 24. | — |
| Oligo- or polyesters with terminal ester of ether groups also comprising α,β-di- carboxylic acid units | Diethylmalonate PPG-600 meth- acrylic acid condensate | 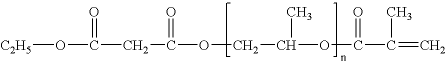 | | — |
| | Ethyl malonate dipropylene glycol n-butyl-ether | 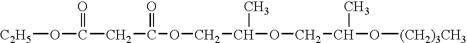 | | — |
| Diesters of 3-oxo- dicarboxylic acids | Dimethyl-3- oxo-glutarate | 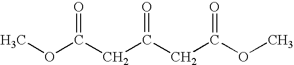 | | 1830-54-2 |
| | Diethyl-3-oxo- glutarate | 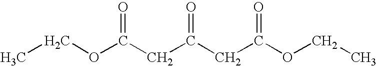 | 17. | 105-50-0 |
| Diesters of α,γ- acetylated di- carboxylic acids | Dimethyl acetyl- succinate | 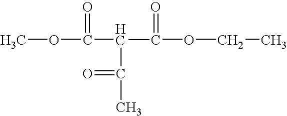 | 10. | 10420-33-4 |
| | Diethyl acetyl- succinate | 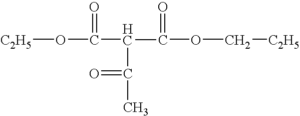 | | |
| α,β-Keto- phosphonates | Dimethyl acetylmethyl phosphonate | 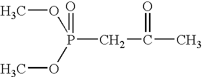 | | 4202-14-6 |
| | Dimethyl phen- acetylmethyl phosphonate | 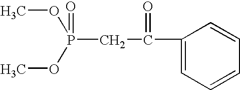 | | |
| α,β-Ketoacid ester phosphonates | Diethyl ethoxycar- bonylmethyl phosphonate | 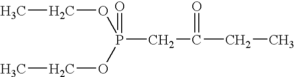 | 11. | 867-13-0 |
| | tert•Butyl P,P-di- methyl phosphono- acetate | 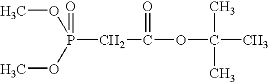 | | 62327-21-3 |
| α,βDiphosphonates | Tetramethyl methylenedi- phosphonate | 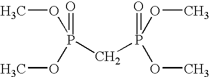 | | |
| | Tetraethyl methylenedi- phosphonate | 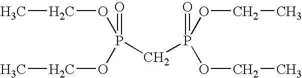 | | 1660-94-2 |

TABLE 2
Cocatalysts incompatible with polyisocyanates

| Compound | | Formula of | Example | |
|---|---|---|---|---|
| group | Cocatalyst | the cocatalyst | for use | CAS No. |
| Salts of derivatives of carboxylic acids comprising acidic —CH₂— or —CH—group | Salts of malaonic acid | 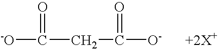 | | 141-82-2 |
| | Barbituric acid or N,N′-malonyl- urea | 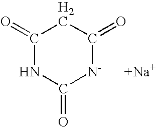 | 1. | 67-52-7 |
| | Potassium salt of monomethyl malonate | 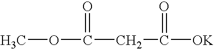 | | 38330-80-2 |
| | Potassium salt of monoethyl malonate | 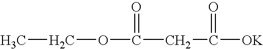 | | 6148-64-7 |
| Other acetoacetic acid, malonic acid and acetylacetone derivatives comprising acidic —CH₂— or —CH—groups | Malonamide | 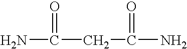 | 13. | 108-13-4 |
| | Diethyl form- amido- malonate | 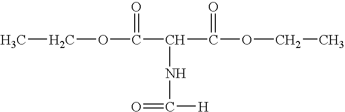 | | 6326-44-9 |
| | Diethyl acetamido- malonate | 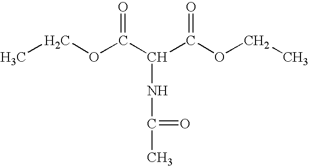 | | 1068-90-2 |
| | Acetoacet- amide | 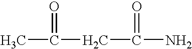 | 12. | 5977-14-0 |
| | N-Methyl-ace- toacetamide | 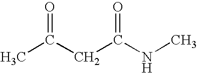 | 15. | 20306-75-6 |
| | N-(2-Hydroxy- ethyl)-aceto- acetamide | 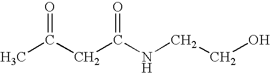 | 21. | 24309-97-5 |
| | Acetoaacet- anilide | 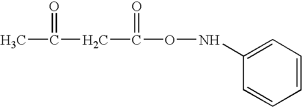 | 13. | 102-01-2 |
| | Diacetoacet- 1,4-phenyl- ene-diamide | 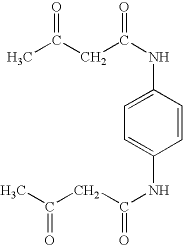 | 22. | 24731-73-5 |
| | Curcumin | 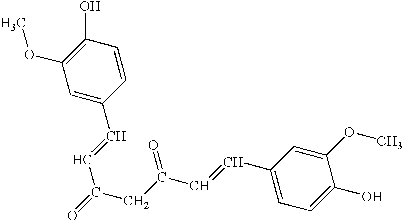 | | 458-37-7 |

TABLE 3

| | | Master batch “A” | | Master batch “B” | | Master batch “C” | | |
|---|---|---|---|---|---|---|---|---|
| Example | | Water glass | AMH comp. | Reactives + | AMH comp. | AMH comp. | Others | Mixing ratio |
| No. | Variant | w/w % | w/w % | additives w/w % | w/w % | w/w % | w/w % | A:B:C v/v |
| Reference 01 | | 100 | nil | 100 | nil | nil | nil | 1:2.5:nil |
| 1 | 1a | 96.13 | 3.87 | 100 | nil | nil | nil | 1:2.5:nil |
| | 1b | 99.61 | 0.39 | 100 | nil | nil | nil | 1:2.5:nil |
| 2-11 | 2a-11a | 100 | nil | 98 | 2.0 | nil | nil | 1:2.5:nil |
| | 2b-11b | 100 | nil | 99.8 | 0.2 | nil | nil | 1:2.5:nil |
| 12-13 | 12a-13a | 100 | nil | Reactives 89.4 | nil | 10.9 | Additives 89.1 | 1:2:0.5 |
| | | | | Additives 10.6 | | | | |
| | 12b-13b | 100 | nil | Reactives 91.6 | nil | 1.1 | Additives 98.9 | 1:2:0.5 |
| | | | | Additives 8.4 | | | | |
| Reference 014 | | 100 | nil | 100 | nil | nil | nil | 1:2:nil |
| 14 | 14a | 96.78 | 3.22 | 100 | nil | nil | nil | 1:2:nil |
| | 14b | 99.68 | 0.32 | 100 | nil | nil | nil | 1:2:nil |
| 15-19 | 15a-19a | 100 | nil | 97.9 | 2.08 | nil | nil | 1:2:nil |
| | 15b-19b | 100 | nil | 99.79 | 0.21 | nil | nil | 1:2:nil |
| 20-21 | 20a-21a | 100 | nil | Reactives 71.8 | nil | 22.7 | Additives 77.3 | 1:1.8:0.2 |
| | | | | Additives 28.2 | | | | |
| | 20b-21b | 100 | nil | Reactives 73.6 | nil | 2.3 | Additives 97.7 | 1:1.8:0.2 |
| | | | | Additives 26.4 | | | | |
| 22 | | 100 | nil | 68 | 42 | nil | nil | 1:4:nil |
| 23 | | 100 | nil | 80 | 20 | nil | nil | 1:3:nil |
| 24 | | 99 | nil | 97.9 | 2.08 | nil | 100 | 1:2:nil |
| 25 | | 100 | nil | 97.87 | 2.13 | nil | 100 | 1:1.8:0.2 |

TABLE 4

| | | Contained in | | | | Ref. pot | | Bending/tensile forces, | |
|---|---|---|---|---|---|---|---|---|---|
| Example | Compounds comprising acidic | master batch | | | Mixing ratio | life, min | | N 1 day/1 week | |
| No. | mobile H | A | B | C | A:B:C, v/v | (a) | (b) | (a) | (b) |
| 1 | None (reference example 01) | − | − | − | 1:2.5:nil | 110 | | 800/2250 | |
| 1 | Barbituric acid sodium salt | + | | | 1:2.5:nil | 95 | — | 1000/2260 | — |
| 2 | Benzoylacetone | | + | | 1:2.5:nil | 1 | 36 | — | 2230/2350 |
| 3 | Zr(IV) acetylacetonate | | + | | 1:2.5:nil | 6 | 13 | 2100/2380 | 2230/2360 |
| 4 | Acetyl-γ-butyrolactone | | + | | 1:2.5:nil | 50 | — | 1300/2520 | — |
| 5 | Benzyl acetoacetate | | + | | 1:2.5:nil | 5 | — | 2120/2400 | — |
| 6 | Ethyl 2-oxo-cyclohexanecarboxylate | | + | | 1:2.5:nil | 75 | — | 1800/2050 | — |
| 7 | Dimethyl malonate | | + | | 1:2.5:nil | 58 | — | 1900/2400 | — |
| 8 | Dimethyl methoxymalonate | | + | | 1:2.5:nil | 90 | — | 2000/2300 | — |
| 9 | Diethyl phenylmalonate | | + | | 1:2.5:nil | 120 | — | 1900/2200 | — |
| 10 | Dimethyl acetylsuccinate | | + | | 1:2.5:nil | 40 | — | 2000/2300 | — |
| 11 | Diethyl-ethoxycarbonyl-methyl-phosphonate | | + | | 1:2.5:nil | 43 | 110 | 2200/2300 | 2120/2350 |
| 12 | Acetoacetamide | | | + | 1:2:0.5 | — | 63 | — | 2100/2240 |
| 13 | Malonamide + acetoacetanilide | | | + | 1:2:0.5 | 6 | 30 | — | 1800/2200 |
| 14 | None (reference example 014) | − | − | − | 1:2:nil | 30 | | 1500/1700 | |
| 14 | N-Methyl-acetoacetamide | + | | | 1:2:nil | — | 15 | — | 1720/1860 |
| 15 | N-Methyl-isobutyrylacetate + | | + | | 1:2:nil | — | 6 | — | 1700/2040 |
| | N,N-diethylacetoacetamide | | | | | | | | |
| 16 | Diethyl oxoglutarate + Lonzamon^{(R)} AAEMA | | + | | 1:2:nil | — | 13 | — | 1760/2070 |
| 17 | tert. Butyl acetoacetate | | + | | 1:2:nil | 8 | — | 1700/2010 | — |
| 18 | Diethyl allylmalonate | | + | | 1:2:nil | 30 | — | 1870/2070 | — |
| 19 | 1,3-Cyclohexanedione | | + | | 1:2:nil | — | 20 | — | 1900/2050 |
| 20 | N-(2-Hydroxyethyl)-aceto-acetamide | | | + | 1:1.8:0.2 | — | 10 | — | 1800/1950 |
| 21 | Diacetoacet-1,4-phenylene-diamide | | | + | 1:1.8:0.2 | — | 8 | — | 1870/2000 |
| 22 | PEG-2000-bis-acetoacetate + Eusolex^{(R)} 9020 | | + | | 1:4:nil | — | 0.5′ | solid foam | |
| 23 | TDI prepolymer + diethylmalonate/ | | + | | 1:3:nil | — | 1.0′ | elastic foam | |
| | neopentyl glycol condensate | | | | | | | | |
| 24 | Diethyl allylmalonate + Lonzamon AAEMA + | | + | | 1:2:nil | 5 | — | 1950/2180 | — |
| | inorganic free radical initiator* | | | | | | | | |
| 25 | Diethyl allylmalonate + Lonzamon AAEMA + | | + | | 1:1.8:0.2 | 6 | — | 2020/2250 | — |
| | organic free radical initiator** | | | | | | | | |

*Contained in master batch A
**Contained in master batch C

What we claim is:

1. A method for the production of polyisocyanate/polysilicic acid based resins with widely variable processability period and setting period by reacting one or more polyisocyanates with water glass optionally in the presence of one or more additives and/or auxiliary agents conventionally used in the production of polyisocyanate/polysilicic acid based resins, characterised in that the reaction is performed in the presence of one or more cocatalysts comprising mobile hydrogen of acidic character, wherein the cocatalyst is a compound comprising a structural unit of 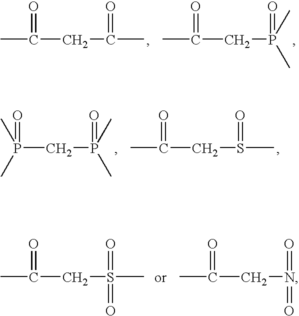
a metal chelate thereof or a derivative thereof in which one of the mobile hydrogens of acidic character is replaced by a substituent X, wherein X represents halo, hydrocarbyl, hydrocarbyl-oxy, hydrocarbyl-carbonyl, hydrocarbyl-oxy-carbonyl or hydrocarbyl-carbonyl-amido group or a combined group formed from two or more of these groups, and the cocatalyst is used in an amount of at least 0.01% by weight calculated for the combined weight of polyisocyate(s) and water glass.

2. A method as claimed in claim 1, characterised in that a compound comprising a 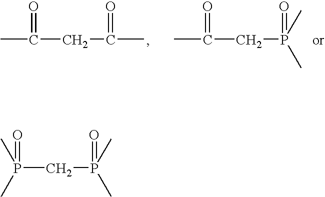
structural unit, a metal chelates thereof and/or a derivative thereof wherein one of the acidic mobile hydrogen is replaced by substituent X is used as cocatalyst.

3. A method as claimed in claim 1 or 2, characterised in that any of the following compounds is used as cocatalyst: α,β-diketones, cyclic α,β-diketones, α,β,γ-triketones, esters of α,β-ketocarboxylic acids, amides of α,β-ketocarboxylic acids, esters of cyclic α,β-ketocarboxylic acids, esters of α,β,β-diketo-monocarboxylic acids, mixed esters of α,β-ketocarboxylic acids and vinylcarboxylic acids formed with glycols, esters of α,β-ketocarboxylic acids formed with polyols, diesters of α,β-dicarboxylic acids, diamides of α,β-dicarboxylic acids, cyclic esters of α,β-dicarboxylic acids, oligo- or polyesters formed from α,β-dicarboxylic acids and polyols, oligo- or polyesters with terminal ester or ether groups also comprising α,β-dicarboxylic acid units, diesters of 3-oxo-dicarboxylic acids, diesters of α,γ-acetylated dicarboxylic acids, α,β-keto-phosphonates, α,β-ketoacid ester phosphonates, α,β-diphosphonates, metal chelates of these compounds, derivatives of these compounds wherein one of the hydrogens of the —CH₂— group is replaced by substituent X, furthermore malonic acid, acetoacetic acid, acetylacetone and derivatives thereof.

4. A method as claimed in any of claims 1 to 3, characterised in that any of the following compounds is used as cocatalyst:
compounds of formula (I)
R—CO—CHY—CO—R¹ (I)
wherein
Y is hydrogen, halo, phenyl, C1-C6 alkyl, C2-C6 alkenyl, C1-C6 alkoxy, phenyl-(C1-C6 alkyl), (C1-C6 alkoxy)-(C1-C6 alkyl), (C1-C6 alkoxy)-carbonyl, formamido, (C1-C6 alkyl)-carbonyl-amido or (C1-C6 alkyl)-carbonyl, and
R and R¹ each stand for
(i) C1-C10 alkyl bearing optionally one or more halo, (C1-C6 alkyl)-carbonyl or (C1-C6 alkoxy)-carbonyl substituent(s),
(ii) phenyl group or a monocyclic heteroaryl group comprising a single hetero atom, each bearing optionally one or more C1-C6 alkyl or C1-C6 alkoxy substituent(s),
(iii) a group of formula —OR², wherein R² stands for hydrogen, a metal atom, phenyl-(C1-C6 alkyl), C2-C10 alkenyl or a C1-C15 alkyl bearing optionally one or more hydroxy, C1-C6 alkoxy, (C1-C6 alkyl)-CO-(C1-C6 alkyl)-CO—O— or (C2-C6 alkenyl)-carbonyl-oxy substituent(s),
(iv) a group of formula —NR³R⁴ wherein R³ and R⁴ each stand for hydrogen, C1-C6 alkyl or hydroxy-(C1-C6 alkyl) or one of them may also represent phenyl wherein the phenyl group may optionally bear an alkyl-carbonyl-alkyl-carbonyl-amido substituent;
(v) C2-C10 alkenyl which may optionally bear a phenyl substituent and the phenyl group may optionally bear a hydroxy and/or C1-C6 alkoxy substituent; or
one of R and R¹ may also represent halo, (C1-C6 alkoxy)-carbonyl, a residue of a polyol wherein the hydroxy groups are esterified and/or etherified, or a group of the formulae —O-(Alk-O)ₘ-CO-Alk¹-CO-Alk², —O-(Alk-O—CO-Alk¹-CO)ₙ-O-Alk², —O-(Alk-O)ₙ-CO-Alk² or —O-(Alk-O)ₙ-(Alk¹-O)ₙ-Alk², and in these latter formulae m is 1-2500, n is 1-60, Alk² is C1-C6 alkyl or C2-C6 alkenyl and Alk and Alk¹ stand for C1-C6 alkylene which may be the same or different; or
R and R¹ may form together a —NH—CO—NH— group, a C2-C4 alkylene chain or a methylenedioxy group, all of which may optionally bear one or more C1-C6 alkyl substituent(s), or
R and Y may form together a C2-C4 alkylene chain or a C2-C4 alkylene-oxy chain, all of which may optionally bear one or more C1-C6 alkyl substituents;
metal chelates of compounds of formula (I) wherein Y is hydrogen and R and R¹ are as defined in points (i) and (ii) above; furthermore
compounds of formulae (II) and (III), 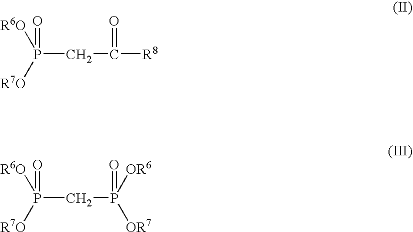
wherein R⁶ and R⁷ each stand for C1-C6,alkyl which may be the same or different and R⁸ represents C1-C6 alkyl, C1-C6 alkoxy or phenyl.

5. A method as claimed in any of claims 1 to 4, characterised in that the cocatalyst is used in an amount of at least 0.05% by weight calculated for the combined weight of polyisocyanate(s) and water glass.

6. A method as claimed in any of claims 1 to 5, characterised in that when any of the cocatalysts, additives and auxiliary agents comprises one or more unsaturated bonds capable of radical polymerization, a free radical initiator is also added to the reaction mixture.

7. A method as claimed in any of claims 1 to 5, characterised in that the cocatalysts which do not bear substituent X or their metal chelates are used in an amount of up to 5% by weight calculated for the combined weight of polyisocyanate(s) and water glass.

8. A method as claimed in any of claims 1 to 5, characterised in that the cocatalysts bearing substituent X are used in an amount of up to 40% by weight calculated for the combined weight of polyisocyanate(s) and water glass.

9. A method as claimed in claim 6, characterised in that the free radical initiator is used in an amount of up to 3% by weight calculated for the combined weight of polyisocyanate(s) and water glass.
